(19) United States
(12) Patent Application Publication
(10) Pub. No.: US 2023/0190097 A1
(43) Pub. Date: Jun. 22, 2023

(54) CATARACT DETECTION AND ASSESSMENT

(71) Applicant: Welch Allyn, Inc., Skaneateles Falls, NY (US)

(72) Inventors: Allen R. Hart, Maryville, TN (US); David L. Kellner, Baldwinsville, NY (US); John A. Lane, Venice, FL (US); Yaolong Lou, Singapore (SG)

(21) Appl. No.: 18/065,428

(22) Filed: Dec. 13, 2022

Related U.S. Application Data
(60) Provisional application No. 63/265,875, filed on Dec. 22, 2021.

(51) Int. Cl. A61B 3/117; G06T 7/00

(52) U.S. Cl. CPC A61B 3/1176; G06T 7/0012; G06T 2207/30041; G06T 2207/10048

(57) ABSTRACT

An eye imager includes a camera having at least one infrared LED. The eye imager captures a sequence of infrared images of an eye using the camera. The eye imager selects an infrared image from the sequence of infrared images. The eye imager determines a cataract is detected in the infrared image and performs an action based on detection of the cataract.

### BACKGROUND

A cataract is a clouding of the eye that is typically caused by the breakdown of normal proteins in the lens of the eye over time. Cataracts can also be caused by injuries to the eye, be present at birth, and in rare cases develop in children, which is often referred to as childhood cataracts. Individuals who smoke, have diabetes, spend long period of time in the sun without sunglasses, and those who take certain medications are more susceptible to cataract development, but most commonly cataracts occur naturally with age.

When cataracts are detected early, certain lifestyle changes can be made to reduce the rate of progression. This is especially true for smokers and diabetics. Unfortunately, many patients do not complete their annual eye exam. Since cataracts progress slowly over time, patients are often not aware that they have cataracts until their vision is severely impacted.

### SUMMARY

In general terms, the present disclosure relates to techniques within an eye imager to screen for cataracts in a non-specialist setting such as a primary care location. Various aspects are described in this disclosure, which include, but are not limited to, the following aspects.

One aspect relates to an eye imager, comprising: a camera having at least one infrared LED; at least one processing device in communication with the camera; and at least one computer readable data storage device storing instructions which, when executed by the at least one processing device, cause the eye imager to: capture a sequence of infrared images of an eye using the camera; select an infrared image from the sequence of infrared images; determine whether a cataract is detected in the infrared image; and perform an action based on detection of the cataract.

Another aspect relates to a method of screening for cataracts, comprising: capturing a sequence of infrared images of an eye; selecting an infrared image from the sequence of infrared images; determining whether a cataract is detected in the infrared image; and performing an action based on detection of the cataract.

Another aspect relates to an eye imager, comprising: at least one processing device; and at least one computer readable data storage device storing instructions which, when executed by the at least one processing device, cause the eye imager to: segment a bright region of a pupil from a dark region of an iris; extract features from the bright region of the pupil; generate a curve based on the features; and detect a cataract based on a comparison of the curve to the cataract profile.

### DESCRIPTION OF THE FIGURES

The following drawing figures, which form a part of this application, are illustrative of the described technology and are not meant to limit the scope of the disclosure in any manner.

FIG. 1 schematically illustrates an example of a system including an eye imager that is operable by a clinician to screen the eyes of a patient for cataracts.

FIG. 2 schematically illustrates an example of a camera in the eye imager of FIG. 1.

FIG. 3 illustrates an example of a method of screening for cataracts performed on the eye imager of FIG. 1.

FIG. 4 illustrates an example of cataract classifications determined from a machine learning model used in an operation of the method of FIG. 3.

FIG. 5 illustrates an example of a method of screening for cataracts by analyzing an image captured by the eye imager of FIG. 1.

FIG. 6 shows an example of a surface type plot generated from pixel intensities for a pupil without cataracts that are extracted from an operation of the method of FIG. 5.

FIG. 7 shows an example of a curve that includes the pixel intensities from FIG. 6 displayed relative to a cataract profile.

FIG. 8 shows an example of a surface type plot generated from pixel intensities for a pupil with cataracts that are extracted from an operation of the method of FIG. 5.

FIG. 9 shows an example of a curve that includes the pixel intensities from FIG. 8 displayed relative to a cataract profile.

FIG. 10 shows another example of a surface type plot generated from pixel intensities for a pupil without cataracts.

FIG. 11 shows another example of a surface type plot generated from pixel intensities for a pupil with cataracts.

FIG. 12 schematically illustrates example components of a computing device of the eye imager of FIG. 1.

### DETAILED DESCRIPTION

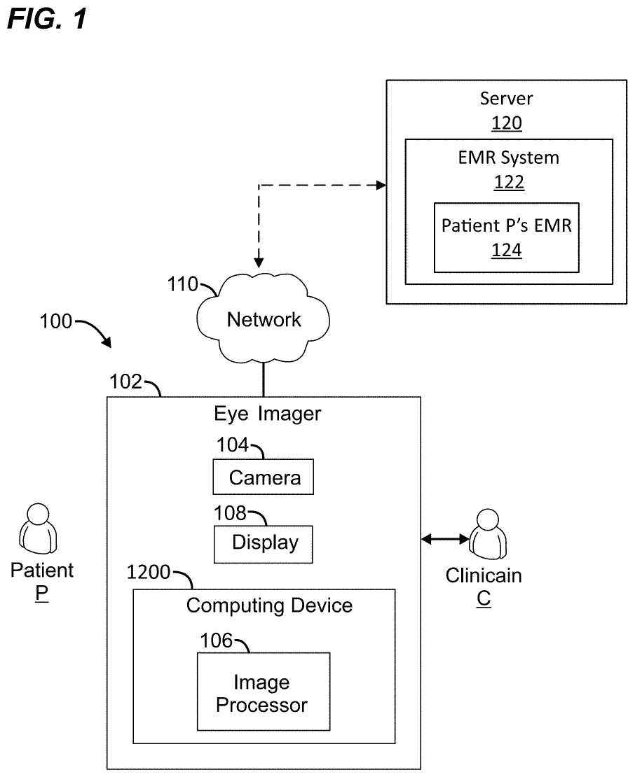

FIG. 1 schematically illustrates an example of a system 100 including an eye imager 102. As shown in FIG. 1, the eye imager 102 is operable by a clinician C to screen the eyes of a patient P for one or more eye diseases and/or conditions. In some examples, the eye imager 102 is a vision screener such as the SPOT® Vision Screener from Hill-Rom Services, Inc. of Batesville, Ind.

In other examples, the eye imager 102 is a fundus imager such as the RetinaVue® 700 Imager from Hill-Rom Services, Inc. of Batesville, Ind. In such examples, the eye imager 102 captures fundus images of the patient P. As used herein, “fundus” refers to the eye fundus, which includes the retina, optic nerve, macula, vitreous, choroid, and posterior pole.

In certain aspects, the eye imager 102 includes components similar to those that are described in U.S. Pat. No. 9,237,846 issued on Jan. 19, 2016, in U.S. Pat. No. 11,096,574, issued on Aug. 24, 2021, and in U.S. Pat. No. 11,138,732, issued on Oct. 5, 2021, which are hereby incorporated by reference in their entireties.

The eye imager 102 can be used by the clinician C to screen, diagnose, and/or monitor the progression of one or more eye diseases and conditions, including retinopathy, macular degeneration, glaucoma, papilledema, and the like. Additionally, the eye imager 102 can be used to screen, diagnose, and/or monitor the progression of cataracts.

In some examples, the clinician C is an eye care professional such as an optometrist or ophthalmologist who uses the eye imager 102 to screen, diagnose, and/or monitor the progression of one or more eye diseases and conditions. In further examples, the clinician C can be a medical professional who is not trained as an eye care professional such as a general practitioner or primary care physician. In such examples, the eye imager 102 can be used to screen for one or more eye diseases and conditions in a primary care medical office.

In further examples, the clinician C can be a non-medical practitioner such as an optician who can help fit eyeglasses, contact lenses, and other vision-correcting devices such that the eye imager 102 can be used to screen for one or more eye diseases and conditions in a retail clinic. In further examples, the eye imager 102 can be used by the patient P as a home device to screen, diagnose, and/or monitor for various types of eye diseases and conditions.

The eye imager 102 can be configured to screen for eye diseases and conditions in a general practice medical office, retail clinic, or patient home by capturing one or more eye images, detecting the presence of one or more conditions in the captured eye images, and providing a preliminary diagnosis for an eye disease/condition or a recommendation to follow up with an eye care professional. In some examples, the eye imager 102 includes software algorithms that can analyze the captured eye images to provide an automated diagnosis based on the detection of conditions in the captured eye images. In such examples, the eye imager 102 can help users who are not trained eye care professionals to screen for one or more eye diseases.

One technique for eye imaging (e.g., of the fundus) requires mydriasis, or the dilation of the patient's pupil, which can be painful and/or inconvenient to the patient P. The eye imager 102 does not require a mydriatic drug to be administered to the patient P before imaging, although the eye imager 102 can image the fundus if a mydriatic drug has been administered.

As shown in FIG. 1, the eye imager 102 includes a computing device 1200 having an image processor 106. The eye imager 102 further includes a camera 104 in communication with the computing device 1200, and a display 108 in communication with the computing device 1200. The camera 104 captures digital images of the eyes of the patient P, and the display 108 displays data, summary reports, and the captured digital images for viewing by the clinician C.

The camera 104 is in communication with the image processor 106. The camera 104 is a digital camera that includes a lens, an aperture, and a sensor array. The lens can be a variable focus lens, such as a lens moved by a step motor, or a fluid lens, also known as a liquid lens. The camera 104 is configured to capture images of the eyes one eye at a time. In other examples, the camera 104 is configured to capture an image of both eyes substantially simultaneously. In such examples, the eye imager 102 can include two separate cameras, one for each eye.

The display 108 is in communication with the image processor 106. In the examples shown in the figures, the display 108 is supported by a housing. In other examples, the display 108 can connect to an image processor that is external of the eye imager 102, such as a separate smartphone, tablet computer, or external monitor. The display 108 functions to display the images produced by the camera 104 in a size and format readable by the clinician C. In some examples, the display 108 is a liquid crystal display (LCD) or active matrix organic light emitting diode (AMOLED) display. In some examples, the display 108 is touch sensitive.

As shown in FIG. 1, the eye imager 102 is connected to a network 110. The eye imager 102 can upload eye images, videos, data, and summary reports to a remote server 120 via the network 110. In some examples, the remote server 120 is a cloud server.

In some examples, the remote server 120 includes an electronic medical record (EMR) system 122 (alternatively termed electronic health record (EHR)). Advantageously, the remote server 120 can automatically store the eye images, videos, data, and summary reports of the patient P in an electronic medical record 124 of the patient P located in the EMR system 122.

In examples where the clinician C is not an eye care professional, the eye images, videos, data, and summary reports stored in the electronic medical record 124 of the patient P can be accessed by an overread clinician who is an eye care professional. Thus, the eye images, videos, data, and summary reports can be accessed and viewed on another device by a remotely located clinician. In such examples, a clinician who operates the eye imager 102 can be different from a clinician who evaluates the eye images, videos, data, and summary reports.

The network 110 may include any type of wireless network, wired network, or any combination of wireless and wired networks. Wireless connections can include cellular network connections. In some examples, a wireless connection can be accomplished directly between the eye imager 102 and an external display device using one or more wired or wireless protocols, such as Bluetooth, Wi-Fi, and the like. Other configurations are possible.

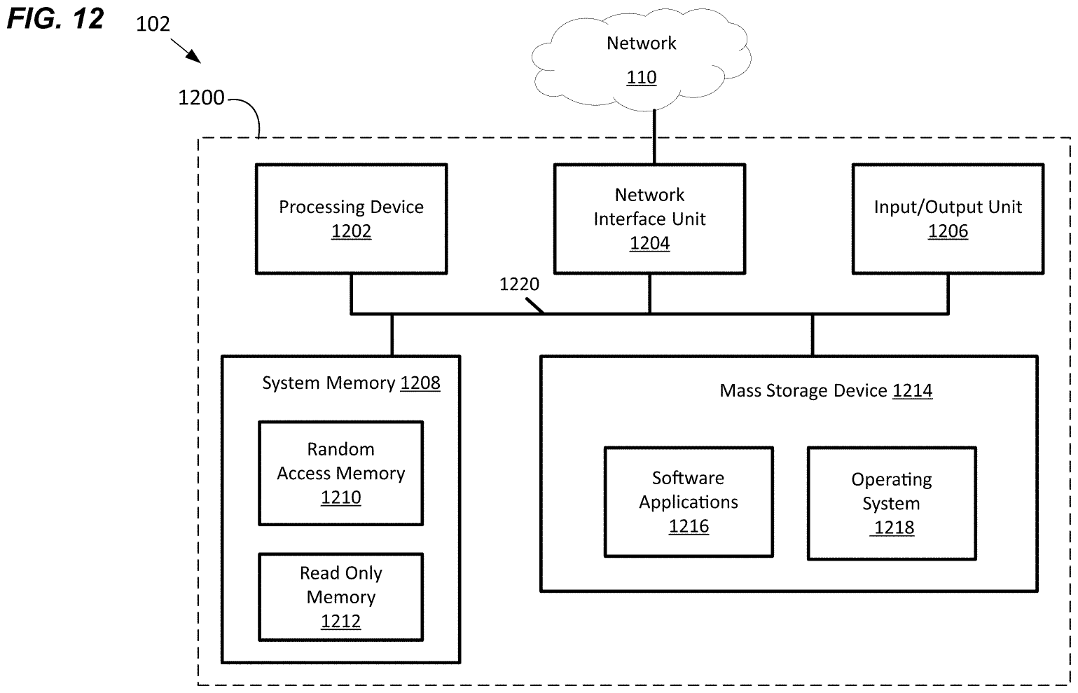

The image processor 106 is coupled to the camera 104 and is configured to communicate with the network 110 and the display 108. The image processor 106 can regulate the operation of the camera 104. Components of an example of the computing device 1200 are shown in more detail in FIG. 12, which will be described further below.

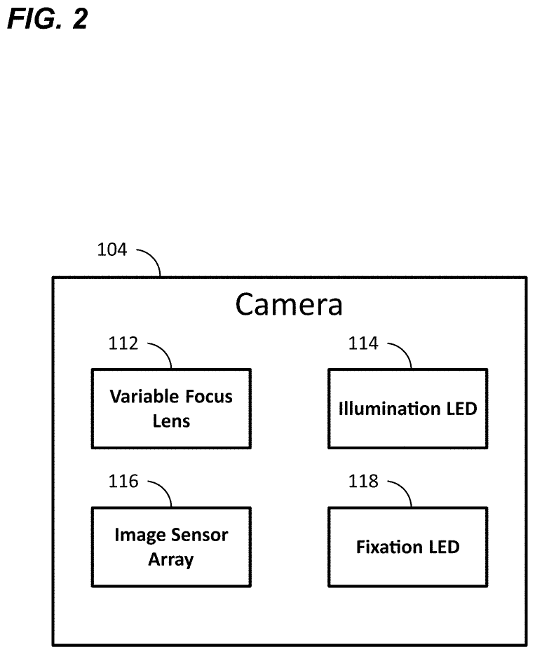

FIG. 2 schematically shows an example of the camera 104. As shown in FIG. 2, the camera 104 includes a variable focus lens 112, an illumination LED assembly 114, an image sensor array 116, and a fixation LED 118. Each component is in electrical communication with the computing device 1200. Alternative examples can include more or fewer components.

In one example, the variable focus lens 112 is a liquid lens. A liquid lens is an optical lens whose focal length can be controlled by the application of an external force, such as a voltage. The lens includes a transparent fluid, such as water or water and oil, sealed within a cell and a transparent membrane. By applying a force to the fluid, the curvature of the fluid changes, thereby changing the focal length. This effect is known as electrowetting.

Generally, a liquid lens can focus between about −10 diopters to about +30 diopters. As used herein, a diopter is a unit of measurement of the optical power of the variable focus lens 112, which is equal to a reciprocal of a focal length measured in meters (e.g., 1 diopter=1 m⁻¹). The focus of a liquid lens can be made quickly, even with large changes in focus. For instance, some liquid lenses can autofocus in tens of milliseconds or faster.

In another example, the variable focus lens 112 is a movable lens controlled by a stepping motor, a voice coil, an ultrasonic motor, or a piezoelectric actuator. Additionally, or as an alternative to moving the variable focus lens 112, a stepping motor can move the image sensor array 116. In such examples, the variable focus lens 112 and/or the image sensor array 116 are oriented normal to an optical axis of the camera 104 and move along the optical axis.

The computing device 1200 coordinates operation of the illumination LED assembly 114 with adjustments of the variable focus lens 112 for capturing one or more images, including fundus images, of the patient P's eyes. In some examples, the illumination LED assembly 114 is a multiple-channel LED, with each LED capable of independent and tandem operation.

The illumination LED assembly 114 includes at least a visible light LED and at least one infrared LED. In some examples, the visible light LED is used for capturing a color eye image (e.g., fundus image), and the infrared LED is used for previewing the image during a preview mode when focusing and locating a field of view, and while minimizing disturbance of the patient P's eyes. For example, the eye imager 102 uses the infrared LED to avoid causing the patient's pupil to constrict while also allowing the clinician C to operate the device in darkness.

The infrared LED can be used to screen for cataracts. The iris is much less reflective than the retina of the eye with respect to infrared light such that when infrared light is directed into the patient P's eye, the infrared light passes through the cornea, the lens, and the vitreous fluid, and then reflects off the retina and back out of the eye. In the resulting infrared image, the pupil is clearly defined by having a bright region which is the reflection of infrared light from the retina, and the region surrounding the pupil (i.e., the iris) is much darker due to the iris being less reflective of infrared light than the retina. An obstruction, such as a cataract, will create an artifact in the bright region of the pupil in the infrared image because the obstruction (e.g., cataract) will block the infrared light from reaching the retina. The darkness and size of the artifact correlates to the opacity and size of the cataract. By scanning through the focus positions of the variable focus lens 112, the focus of the artifact will also change which can be used to indicate the depth of the artifact (e.g., cataract) that is obstructing the path of the infrared light.

The fixation LED 118 produces a light to guide the patient's P eye for alignment. The fixation LED 118 can be a single color or multicolor LED. For example, the fixation LED 118 can produce a beam of light that appears as a dot when the patient P looks into the housing.

The image sensor array 116 receives and processes the light from the illumination LED assembly 114 that is reflected by the patient P's eye. In some examples, the image sensor array 116 is a complementary metal-oxide semiconductor (CMOS) sensor array, also known as an active pixel sensor (APS), or a charge coupled device (CCD) sensor. The image sensor array 116 includes photodiodes that have a light-receiving surface and have substantially uniform length and width. During exposure, the photodiodes convert the incident light to a charge that is used by the image processor 106 (see FIG. 1) to produce an image of the eye.

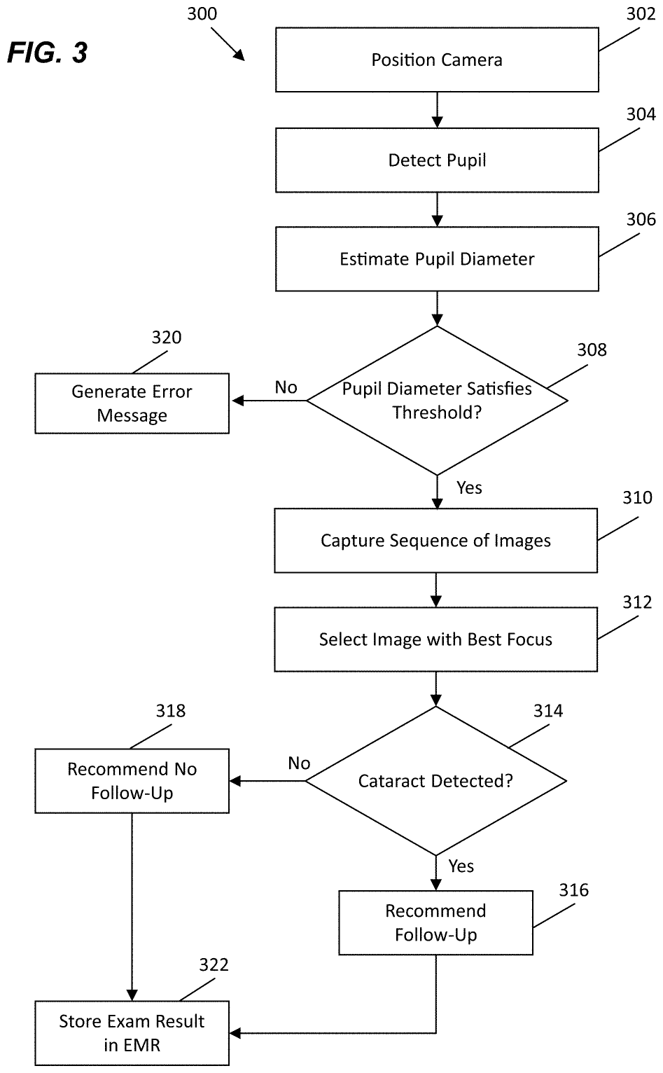

FIG. 3 illustrates an example of a method 300 of screening for cataracts performed on the eye imager 102. The method 300 includes an operation 302 of positioning the camera 104 to align a center of a field of view of the camera 104 with a pupil of an eye of the patient P. The method 300 can be performed for each eye of the patient P. In some examples, the camera 104 is positioned using components of the system described in U.S. Pat. No. 10,993,613, issued on May 4, 2021, which is herein incorporated by reference in its entirety.

Next, the method 300 includes an operation 304 of detecting the pupil. In certain examples, the pupil is detected using algorithms similar to the ones described in U.S. Pat. No. 10,136,804, issued on Nov. 27, 2018, and in U.S. patent application Ser. No. 17/172,827, filed on Feb. 10, 2021, which are hereby incorporated by reference in their entireties.

Next, the method 300 includes an operation 306 of estimating a diameter of the pupil detected from operation 304. In some examples, the diameter of the pupil is estimated using histogram circle detection which allows detection of circles when obstructed.

Next, the method 300 includes an operation 308 of determining whether the pupil diameter (estimated from operation 306) satisfies a threshold size. When the pupil diameter does not satisfy the threshold size such that the pupil is too small for evaluation (i.e., “No” in operation 308), the method 300 can return an error message and terminate at operation 320.

When the pupil diameter satisfies the threshold size such that the pupil is sufficiently large enough for evaluation (i.e., “Yes” in operation 308), the method 300 proceeds to an operation 310 of capturing a sequence of images using the infrared LED of the illumination LED assembly 114. In some examples, the sequence of images are captured by scanning through various focus positions of the variable focus lens 112 to capture images under different focal lengths (e.g., diopters). As will be described in more detail, the different focal lengths can be used to estimate a depth of one or more cataracts when detected in the pupil region.

Next, the method 300 includes an operation 312 of selecting an image from the sequence of images captured in operation 310 that has a best focus. In certain examples, an image with the best focus is determined by identifying the image with a highest standard deviation in Laplacian distribution of pixels. Operation 312 identifies the most focused image such that artifacts present in the bright region of the pupil can be more easily identified.

Next, the method 300 includes an operation 314 of determining whether a cataract is detected in the selected infrared image. Operation 314 includes identifying whether there are any artifacts in the bright region of the pupil from the image selected in operation 312. As described above, the iris is much less reflective than the retina such that when infrared light is directed into the eye, the pupil is clearly defined in a resulting infrared image by a bright region (i.e., reflective retinal tissue) surrounded by the iris (i.e., not reflective tissue). Any obstruction on the pupil that is not clear, such as a cataract, creates an artifact in the bright region because it will block the infrared light from reaching the retina. The darkness and size of the artifact correlates to the opacity and size of the obstruction (e.g., cataract).

In certain examples, operation 314 includes segmenting the artifact from the bright region of the pupil. The contour of the artifact can be used to determine dimensional aspects of the cataract, including a surface area. In some examples, a score is calculated based on a ratio of the surface area of the artifact to the surface area of the bright region of the pupil.

In some examples, a cataract is detected in operation 314 based on whether any artifacts are found in the bright region of the pupil. In further examples, a cataract is detected in operation 314 when the score (i.e., calculated based on a ratio of the surface area of the artifact to the surface area of the bright region of the pupil) exceeds a predetermined threshold.

In further examples, a cataract is detected in operation 314 using a machine learning model that confirms whether the artifact is a cataract (i.e., “Yes” at operation 314) or is not a cataract (i.e., “No” at operation 314). In further examples, a machine learning model is used in operation 314 to classify the detected cataract under one or more types of cataracts including an early-onset cataract, a nuclear cataract, a cortical cataract, and a posterior capsular cataract.

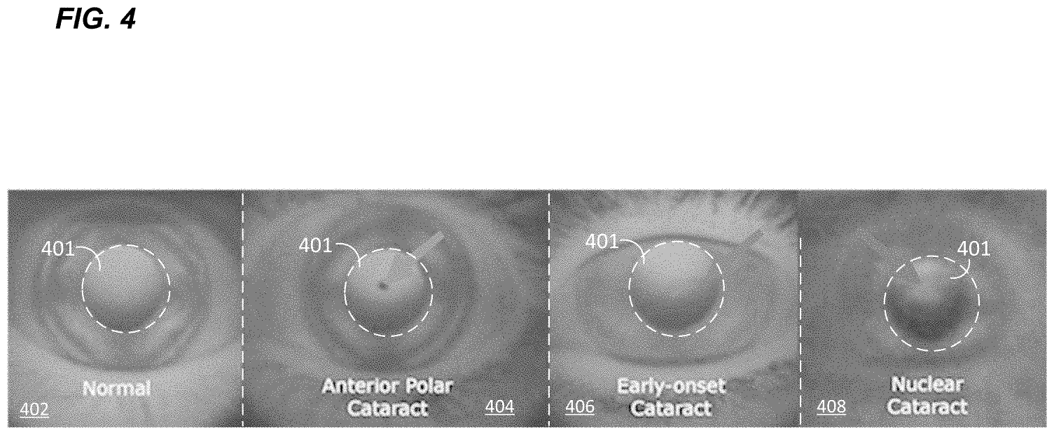

FIG. 4 illustrates an example of cataract classifications determined from the machine learning models used in the operation 314 of the method 300. As shown in FIG. 4, a first image 402 shows that no cataracts are detected inside a bright region 401 of the pupil. In the first image 402, the result of operation 314 is a negative test result (i.e., no cataract detected).

As further shown in FIG. 4, operation 314 can classify a detected cataract into one or more types. For example, a second image 404 identifies an anterior polar cataract inside the bright region 401, a third image 406 identifies an early onset cataract inside the bright region 401, and a fourth image 408 identifies a nuclear cataract inside the bright region 401.

Referring back to FIG. 3, when a cataract is detected (i.e., “Yes” at operation 314), method 300 proceeds to an operation 316 of recommending a follow-up. The recommendation in operation 316 can be displayed on the display 108 of the eye imager 102. In some examples, the recommendation in operation 316 includes dimensions of the detected cataract (e.g., size, density, and depth), a score (i.e., based on a ratio of the surface area of the artifact to the surface area of the bright region of the pupil), and/or a classification of the cataract (e.g., an early-onset cataract, a nuclear cataract, a cortical cataract, and a posterior capsular cataract).

In examples where the method 300 is performed on the eye imager 102 when used by an eye care professional such as an optometrist or ophthalmologist, operation 316 can include recommending a follow-up to perform additional screening tests for cataracts such as a fully dilated eye exam and using a slit lamp. In examples where the method 300 is performed on the eye imager 102 when used by a medical professional who is not trained as an eye care professional such as a primary care physician, operation 316 can include recommending a follow-up with an eye care professional such as an optometrist or ophthalmologist.

When a cataract is not detected (i.e., “No” at operation 314), the method 300 can proceed to an operation 318 of recommending that a follow-up is not needed. For example, the method 300 can include a recommendation that additional screening tests are not needed or a follow-up with a trained eye care professional such as an optometrist or ophthalmologist is not needed. In this manner, the method 300 is performed on the eye imager 102 to screen for cataracts in an efficient and effective way without having to perform invasive tests or exams such as a fully dilated eye exam. Additionally, the method 300 when performed on the eye imager 102 in a non-specialist setting such as in a primary care location can compensate for patients who are unable to complete their annual eye exam. Advantageously, the method 300 when performed on the eye imager 102 can inform a larger patient population to make lifestyle changes to reduce the rate of cataract progression, or to seek treatment such as surgery.

The method 300 further includes an operation 322 of storing the test result (e.g., the positive test result from operation 316 or the negative test result from operation 318) in the electronic medical record 124 of the patient P to maintain a history of cataract screening for the patient P. In examples where the test result in operation 316 includes a dimension, a score, and/or a classification of the detected cataract, operation 322 can include storing the dimension, the score, and/or the classification of the detected cataract in the electronic medical record 124 of the patient P to monitor progression of the detected cataract over time.

The method 300 can be repeated to screen for cataracts in each eye of the patient P. For example, the method 300 can be performed for a first eye of the patient P (e.g., the left eye), and the method 300 can be repeated for a second eye of the patient P (e.g., the right eye).

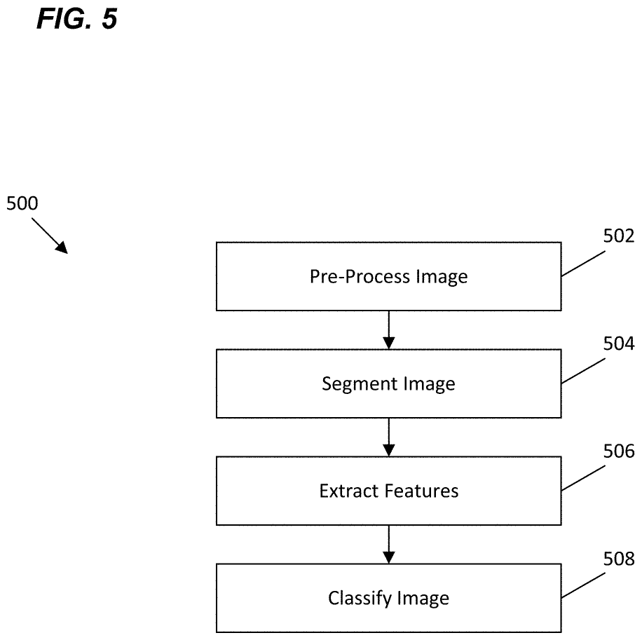

FIG. 5 illustrates an example of a method 500 of screening for cataracts by analyzing an image captured by the eye imager 102. The method 500 is performed on the eye imager 102 to classify an image as not having a cataract (i.e., a negative test result), or to classify an image as having at least one cataract (i.e., a positive test result). Also, the method 500 can be performed on the eye imager 102 to classify a type of detected cataract such as an early-onset cataract, a nuclear cataract, a cortical cataract, and a posterior capsular cataract. Also, the method 500 can be performed to estimate a size, density, and depth of a detected cataract.

As shown in FIG. 5, the method 500 includes an operation 502 of pre-processing an image captured by the camera 104 of the eye imager 102. In some examples, the image is an infrared image captured by using the infrared LED of the illumination LED assembly 114. Operation 502 can include cropping the image, filtering the image, and color processing.

Next, the method 500 includes an operation 504 of segmenting the pre-processed image. For example, operation 504 can include segmenting the bright region that defines the pupil in the infrared image from the dark region that defines the iris in the infrared image.

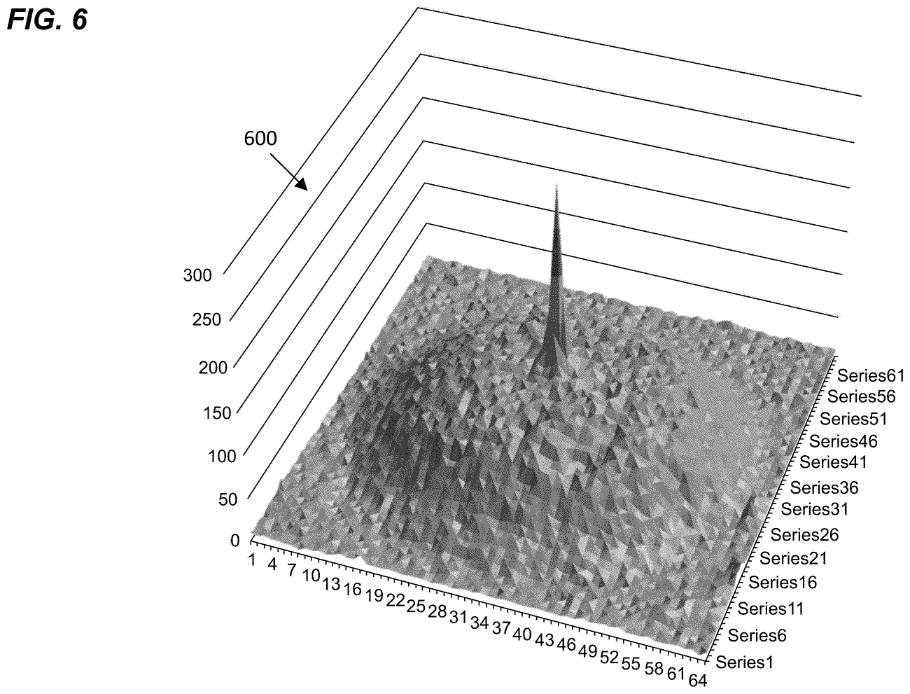

Next, the method 500 includes an operation 506 of extracting features from the segmented bright region. Operation 506 can include extracting pixel intensity values, extracting color values, and extracting texture values. The extracted features can be used to generate surface type plots and graphs of the pupil such as the plots and graphs shown in FIGS. 6-11.

Next, the method 500 includes an operation 508 of classifying the image based on the features extracted in operation 506. In some examples, a machine learning model can use the features extracted in operation 506 to confirm whether a cataract is present or not. In further examples, a machine learning model can use the features extracted in operation 506 to classify the detected cataract under one or more types of cataracts including an early-onset cataract, a nuclear cataract, a cortical cataract, and a posterior capsular cataract.

FIG. 6 shows an example of a surface type plot 600 generated from pixel intensities for a pupil without cataracts that are extracted from operation 506 of the method 500. The pixel intensities represent brightness from reflection of the infrared light. As shown in the example provided in FIG. 6, the pixel intensities of a pupil without cataracts form a convex curve with pixels having a highest intensity in the center of the bright region of the pupil.

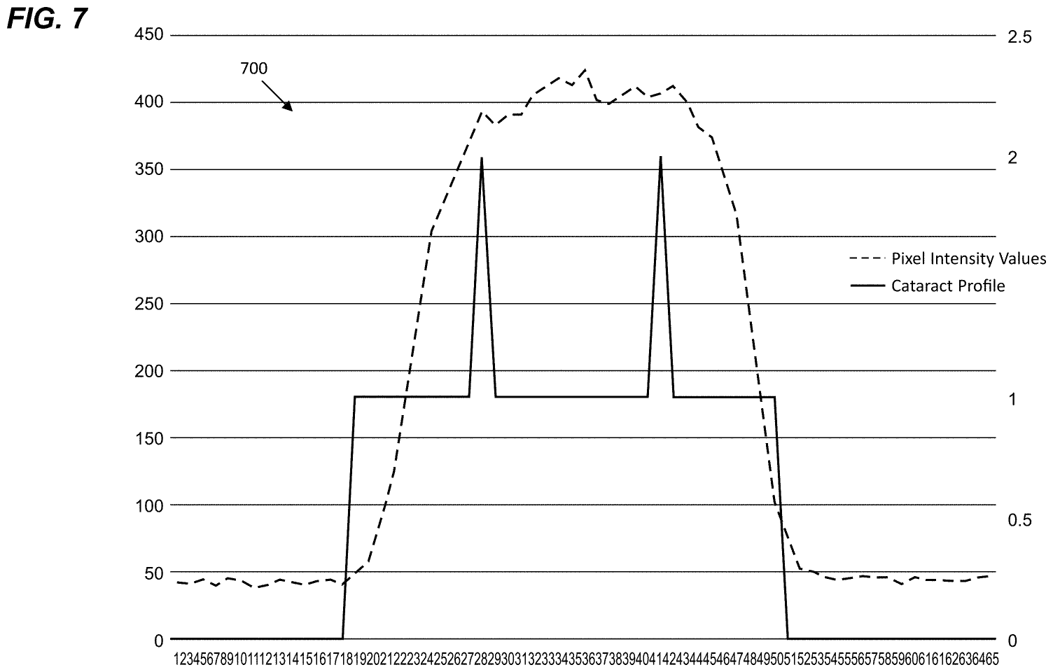

FIG. 7 shows an example of a curve 700 of the pixel intensities from FIG. 6 displayed relative to a cataract profile. In some examples, the cataract profile is predefined and stored in a memory device of the eye imager 102. In the example shown in FIG. 7, the pixel intensities do not correlate to the cataract profile. Accordingly, in this example, operation 508 can generate a negative test result that indicates presence of a cataract is not detected because the pixel intensities do not correlate to the cataract profile.

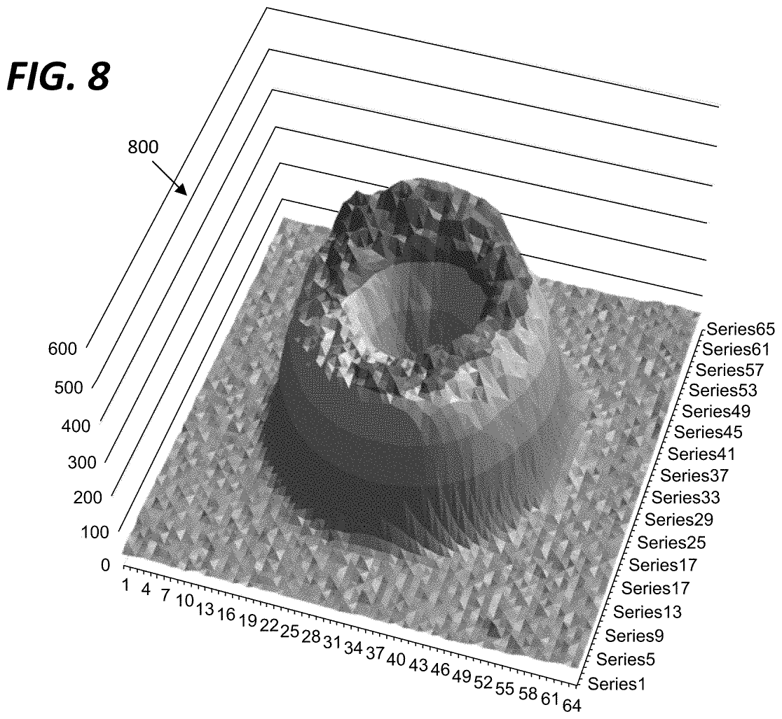

FIG. 8 shows an example of a surface type plot 800 generated from pixel intensities for a pupil with cataracts that are extracted from operation 506 of the method 500. In FIG. 8, the pixel intensities of a pupil with cataracts form a concave curve with pixels having a lower intensity in the center of the bright region of the pupil.

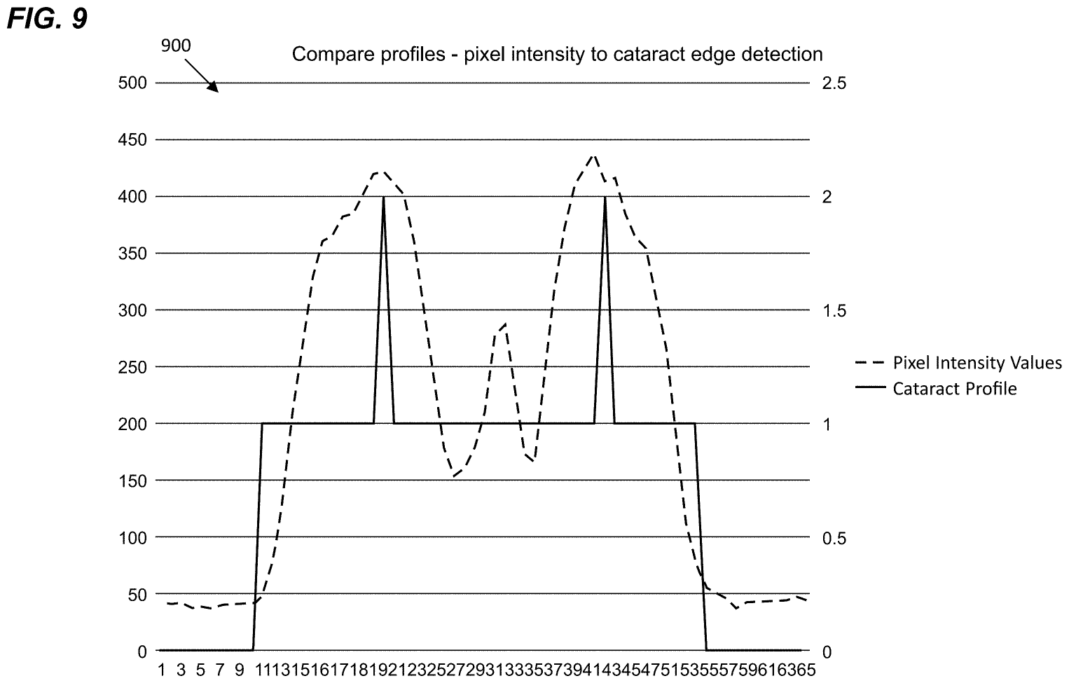

FIG. 9 shows an example of a curve 900 that includes the pixel intensities from FIG. 8 displayed relative to a cataract profile. As described above, the cataract profile can be predefined and stored in a memory device of the eye imager 102. In the example shown in FIG. 9, the pixel intensities correlate to the cataract profile. Accordingly, in this example, operation 508 can generate a positive test result that indicates detection of a cataract because the pixel intensities correlate to the cataract profile.

In view of FIGS. 6-9, the pixel intensities extracted from operation 506 can be used in operation 508 to determine whether a pupil has a cataract, and to classify the cataract. For example, each type of cataract (e.g., a nuclear cataract, a cortical cataract, and a posterior capsular cataract) may have a unique cataract profile. Thus, the curve of the pixel intensities is compared to a predefined cataract profile (see FIGS. 7 and 9) to determine whether a cataract is present, and to classify the cataract based on the predefined cataract profile.

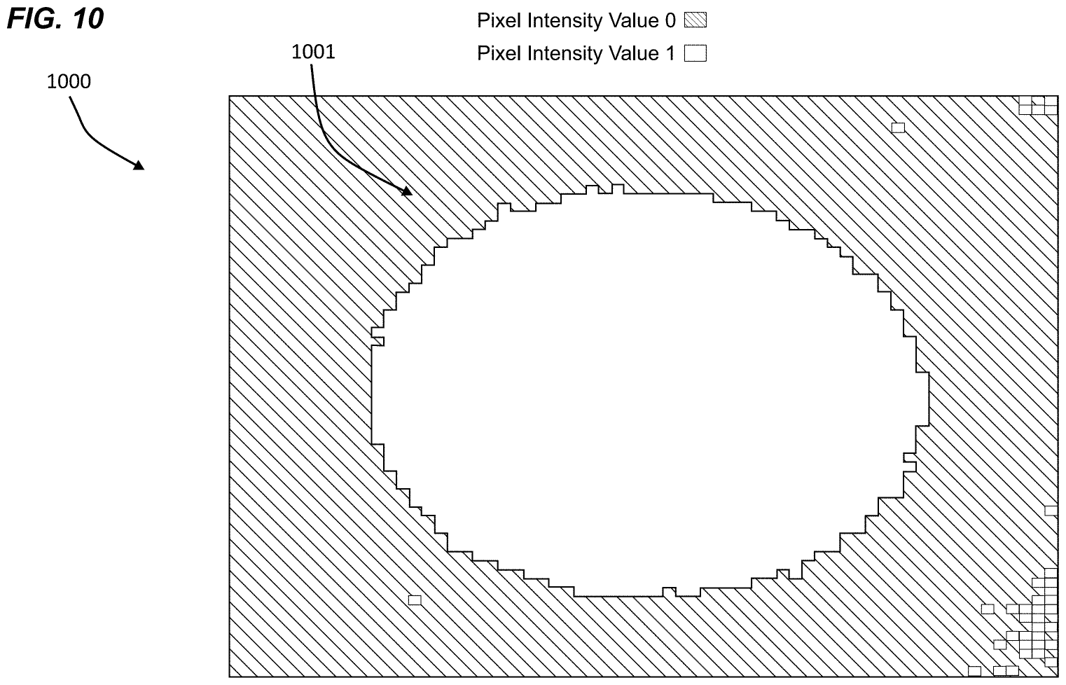

FIG. 10 shows another example of a surface type plot generated from pixel intensities for a pupil without cataracts. In FIG. 10, the glean from the pupil has been removed. As shown in FIG. 10, the pixel intensity values inside the bright region of the pupil are uniform.

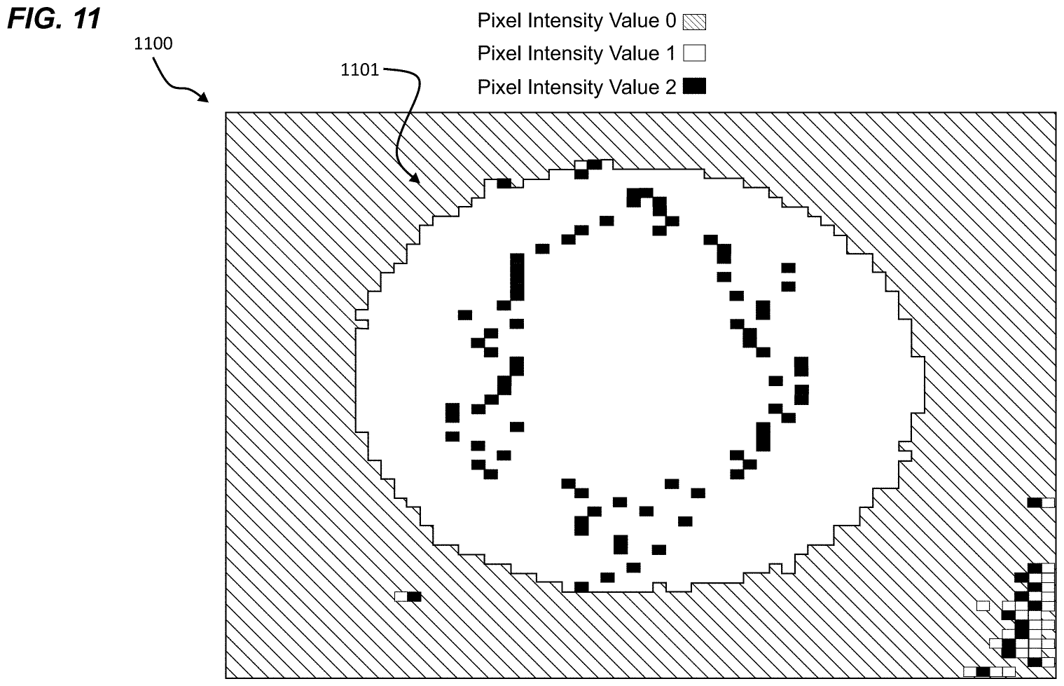

FIG. 11 shows another example of a surface type plot generated from pixel intensities for a pupil with cataracts. In FIG. 11, the glean from the pupil has been removed. In contrast to FIG. 10, the pixel intensity values in FIG. 11 have different values. This indicates that a cataract is present. A pattern from the pixel intensity values can be identified for classifying the cataract such as by matching the pattern to a cataract profile associated with a certain type of cataract.

In further examples, the eye imager 102 can screen for cataracts by binocular imaging. For example, the eye imager 102 can compare images of the left and right eyes to determine whether there are any dissimilarities between the images. This is because cataracts do not typically develop symmetrically in both eyes. Thus, dissimilarities between the left and right eyes may indicate the presence of a cataract in at least one of the eyes.

In further examples, the eye imager 102 can screen for cataracts by applying color filters. For example, the eye imager 102 can switch between one or more color filters when capturing a sequence of images. Color can provide a further indicator of whether a cataract is present because a cataract has a different color (e.g., white) than the color of the pupil (e.g., black). The color contrast in the pupil area can be measured to detect a cataract.

FIG. 12 schematically illustrates example components of the computing device 1200 of the eye imager 102. As shown in FIG. 12, the computing device 1200 includes at least one processing device 1202, a system memory 1208, and a system bus 1220 coupling the system memory 1208 to the at least one processing device 1202. The at least one processing device 1202 is an example of a processor such as a central processing unit (CPU) or microcontroller.

The system memory 1208 is an example of a computer readable data storage device that stores software instructions that are executable by the at least one processing device 1202. The system memory 1208 includes a random-access memory (“RAM”) 1210 and a read-only memory (“ROM”) 1212. Input/output logic containing the routines to transfer data between elements within the eye imager 102, such as during startup, is stored in the ROM 1212.

The computing device 1200 of the eye imager 102 can include a mass storage device 1214 that is able to store software instructions and data. The mass storage device 1214 can be connected to the at least one processing device 1202 through a mass storage controller connected to the system bus 1220. The mass storage device 1214 and associated computer-readable data storage medium provide non-volatile, non-transitory storage for the eye imager 102.

Although the description of computer-readable data storage media contained herein refers to a mass storage device, the computer-readable data storage media can be any non-transitory, physical device or article of manufacture from which the device can read data and/or instructions. The mass storage device 1214 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The eye imager 102 can operate in a networked environment through connections to remote network devices connected to the network 110. The eye imager 102 connects to the network 110 through a network interface unit 1204 connected to the system bus 1220. The network interface unit 1204 can also connect to other types of networks and remote systems.

The eye imager 102 can also include an input/output controller 1206 for receiving and processing input from a number of input devices such as a touchscreen display. Similarly, the input/output controller 1206 may provide output to a number of output devices.

The mass storage device 1214 and the RAM 1210 can store software instructions and data. The software instructions can include an operating system 1218 suitable for controlling the operation of the eye imager 102. The mass storage device 1214 and/or the RAM 1210 also store software instructions 1216, that when executed by the at least one processing device 1202, cause the eye imager 102 to provide the functionalities discussed in this document.

The various embodiments described above are provided by way of illustration only and should not be construed to be limiting in any way. Various modifications can be made to the embodiments described above without departing from the true spirit and scope of the disclosure.

What is claimed is:

1. An eye imager, comprising:
a camera having at least one infrared LED;
at least one processing device in communication with the camera; and
at least one computer readable data storage device storing instructions which, when executed by the at least one processing device, cause the eye imager to:
capture a sequence of infrared images of an eye using the camera;
select an infrared image from the sequence of infrared images;
determine whether a cataract is detected in the infrared image; and
perform an action based on detection of the cataract.

2. The eye imager of claim 1, wherein the infrared image is selected by identifying an image with a highest standard deviation in Laplacian distribution of pixels.

3. The eye imager of claim 1, wherein the action includes generate a recommendation to follow up with an eye care professional.

4. The eye imager of claim 1, wherein the cataract is detected by identifying an artifact in a bright region of a pupil in the infrared image.

5. The eye imager of claim 4, wherein the instructions, when executed by the at least one processing device, further cause the eye imager to:
use a machine learning model to confirm the artifact is a type of cataract.

6. The eye imager of claim 4, wherein the instructions, when executed by the at least one processing device, further cause the eye imager to:
use a machine learning model to classify the artifact as a type of cataract selected from the group consisting of an early-onset cataract, a nuclear cataract, a cortical cataract, and a posterior capsular cataract.

7. The eye imager of claim 4, wherein the instructions, when executed by the at least one processing device, further cause the eye imager to:
segment the artifact from the bright region of the pupil;
determine a surface area of the artifact; and
calculate a score based on a ratio of the surface area of the artifact to a surface area of the bright region of the pupil, and wherein the cataract is detected when the score exceeds a predetermined threshold.

8. The eye imager of claim 7, wherein the instructions, when executed by the at least one processing device, further cause the eye imager to:
store the score in an electronic medical record of a patient for monitoring progression of the cataract over time.

9. The eye imager of claim 1, wherein the instructions, when executed by the at least one processing device, further cause the eye imager to:
generate a curve of pixel intensities inside a bright region of a pupil.

10. The eye imager of claim 9, wherein the instructions, when executed by the at least one processing device, further cause the eye imager to:
store a cataract profile for at least one type of cataract; and
classify the cataract as belonging to the at least one type of cataract by comparing the curve of pixel intensities to the cataract profile.

11. A method of screening for cataracts, comprising:
capturing a sequence of infrared images of an eye;
selecting an infrared image from the sequence of infrared images;
determining whether a cataract is detected in the infrared image; and
performing an action based on detection of the cataract.

12. The method of claim 11, wherein the infrared image is selected by identifying an image with a highest standard deviation in Laplacian distribution of pixels.

13. The method of claim 11, wherein the action includes generate a recommendation to follow up with an eye care professional.

14. The method of claim 11, wherein the cataract is detected by identifying an artifact in a bright region of a pupil in the infrared image.

15. The method of claim 14, further comprising:
segmenting the artifact from the bright region of the pupil;
determining a surface area of the artifact;
calculating a score based on a ratio of the surface area of the artifact to a surface area of the bright region of the pupil; and
detecting the cataract when the score exceeds a predetermined threshold.

16. The method of claim 11, further comprising:
storing a cataract profile for at least one type of cataract; and
classifying the cataract as belonging to the at least one type of cataract by comparing a curve of pixel intensities to the cataract profile.

17. An eye imager, comprising:
at least one processing device; and
at least one computer readable data storage device storing instructions which, when executed by the at least one processing device, cause the eye imager to:
segment a bright region of a pupil from a dark region of an iris;
extract features from the bright region of the pupil;
generate a curve based on the features; and
detect a cataract based on a comparison of the curve to a cataract profile.

18. The eye imager of claim 17, wherein the features extracted from the bright region of the pupil are pixel intensity values.

19. The eye imager of claim 17, wherein the instructions, when executed by the at least one processing device, further cause the eye imager to:
classify the cataract based on the comparison of the curve to the cataract profile, wherein the cataract classified as a type of cataract selected from the group consisting of an early-onset cataract, a nuclear cataract, a cortical cataract, and a posterior capsular cataract.

20. The eye imager of claim 17, wherein the instructions, when executed by the at least one processing device, further cause the eye imager to:
use a machine learning model to confirm classification of the cataract.
